(19) United States
(12) Patent Application Publication
(10) Pub. No.: US 2024/0216405 A1
(43) Pub. Date: Jul. 4, 2024

(54) MOUSE MODEL AND TREATMENT OF HEREDITARY INCLUSION BODY MYOPATHY

(71) Applicant: HIBM Research Group, Inc., Chatsworth, CA (US)

(72) Inventors: Daniel Kohan DARVISH, Sherman Oaks, CA (US); Yadira VALLES-AYOUB, Woodland Hills, CA (US)

(21) Appl. No.: 18/514,227

(22) Filed: Nov. 20, 2023

Related U.S. Application Data
(63) Continuation of application No. 17/152,471, filed on Jan. 19, 2021, now abandoned.
Application No. 17/152,471 is a continuation of application No. 15/479,761, filed on Apr. 5, 2017, now abandoned.
Application No. 15/479,761 is a continuation of application No. 14/676,312, filed on Apr. 1, 2015, now abandoned.
Application No. 14/676,312 is a continuation of application No. 12/128,517, filed on May 28, 2008, now Pat. No. 9,018,174.

(51) Int. Cl. A61K 31/7012; A01K 67/0275; A01K 67/0278; A61D 19/04; C12N 9/12; C12N 9/90; C12Q 1/6883; G01N 33/50; G01N 33/573

(52) U.S. Cl. CPC A61K 31/7012; A01K 67/0275; A01K 67/0278; A61D 19/04; C12N 9/1205; C12N 9/90; C12Q 1/6883; G01N 33/5038; G01N 33/573; A01K 2207/15; A01K 2217/00; A01K 2217/052; A01K 2217/072; A01K 2227/105; A01K 2267/03; A01K 2267/0306; A01K 2267/0318; C12Q 2600/106; C12Q 2600/156; C12Y 207/0106; C12Y 501/03002; G01N 2333/91215; G01N 2333/99; G01N 2500/10

(57) ABSTRACT

Disclosed herein are methods of treating HIBM in a subject comprising identifying subject in need thereof; and administering to the subject a compound, or a pharmaceutically acceptable salt, ester, amide, glycol, peptidyl, or prodrug thereof, wherein the compound is a compound that is biosynthesized in a wild type individual along a biochemical pathway between glucose and sialic acid, inclusive. Also disclosed herein are vectors comprising a nucleic acid sequence that encodes a polypeptide having at least 80% sequence identity to the sequence set forth in SEQ ID NO:2, recombinant cells comprising these vectors, and recombinant animals comprising the cells. In addition, methods of identifying a compound having therapeutic effect for HIBM are disclosed.

### FIELD OF THE INVENTION

The present invention is in the field of treatment of Hereditary Inclusion Body Myopathy and genetically modified mice as test models for the same.

### BACKGROUND OF THE DISCLOSURE

Hereditary Inclusion Body Myopathy (HIBM, MIM 600737) is an autosomal recessive neuromuscular disorder characterized by adult onset, slowly progressive muscle weakness and atrophy. Serum creatine kinase levels are normal to slightly elevated and electromyograms show either a myopathic or a neuropathic pattern. Histologically, muscle fibers degenerate and develop cytoplasmic rimmed vacuoles and cytoplasmic or nuclear filamentous inclusions. No therapy currently exists for HIBM.

The myopathy results from mutations in GNE gene, coding for the bifunctional enzyme UDP-N-acetylglucosamine (GlcNAc) 2-epimerase/N-acetylmannosamine (ManNAc) kinase (GNE/MNK). A GNE founder mutation (M712T) was originally described in Persian-Jewish HIBM families, but numerous other mutations in GNE are now reported in patients worldwide. GNE/MNK is ubiquitously expressed and catalyzes the first two committed, rate-limiting steps in the biosynthesis of N-acetylneuraminic acid (Neu5Ac, sialic acid). The enzyme is feedback-inhibited by the downstream product, CMP-Neu5Ac. Neu5Ac is the most abundant mammalian sialic acid and is typically found as the terminal sugar on glycoconjugates, where it plays a role in a variety of cellular signaling functions. HIBM-associated GNE mutations, result in reduced activity of both GlcNAc 2-epimerase and ManNAc kinase activities; these decrements are considered responsible for reduced production of sialic acid.

The pathologic mechanism of the eventual muscle fiber degeneration of HIBM remains unknown. However, evidence suggests that decreased availability of sialic acid in muscle causes hyposialylation of muscle glycoproteins, whether involving glycans in general, O-linked glycans, polysialic acid on neural cell adhesion molecule (PSA-NCAM), or specific O-mannosylated glycosyl residues on a-dystroglycan. The O-mannosylated residues on α-dystroglycan govern interactions of α-dystroglycan with extracellular matrix proteins, and their deficiency is responsible for several congenital muscular dystrophies, including Walker-Warburg syndrome and Muscle-Eye-Brain discase.

While the above pathways that are implicated in the disease are known, no treatment for the disease has been found to date. Therefore, there is a need in the art for animal models in which the disease can be studied, and treatment regimens that can ameliorate the effects of the disease.

### SUMMARY OF THE INVENTION

Disclosed herein is a vector comprising a nucleic acid sequence that encodes a polypeptide having at least 80% sequence identity to the sequence set forth in SEQ ID NO:2. In some embodiments, the polypeptide has a sequence selected from the group consisting of set forth in SEQ ID NO:2 through SEQ ID NO:19.

Also disclosed herein is a recombinant cell comprising the vector wherein the vector comprises a nucleic acid sequence that encodes a polypeptide having at least 80% sequence identity to the sequence set forth in SEQ ID NO:2. In some of these embodiments, the polypeptide has a sequence selected from the group consisting of set forth in SEQ ID NO:2 through SEQ ID NO:19. In certain embodiments, the cell is a stem cell, which can be an embryonic stem cell. Some of the stem cells are murine.

Further, disclosed herein is a recombinant animal where the animal has a cell that expresses a polypeptide having at least 80% sequence identity to the sequence set forth in SEQ ID NO:2. In some embodiments, the animal is made by the process of producing a vector comprising a nucleic acid sequence that encodes a polypeptide having at least 80% sequence identity to the sequence set forth in SEQ ID NO:2; producing a recombinant mammalian embryonic stem cell by infecting a mammalian embryonic stem cell with the vector; selecting the embryonic stem cell line that has undergone homologous recombination to incorporate the vector sequence at a desired genomic locus; planting the recombinant stem cell in an embryo; implanting the embryo in a female animal; and allowing the implanted embryo to mature into a fully formed fetus and be born from the female animal. In some of these embodiments, the polypeptide has a sequence selected from the group consisting of set forth in SEQ ID NO:2 through SEQ ID NO:19. In certain embodiments, the animal comprises two alleles of the gene that encodes a polypeptide having a sequence set forth in SEQ ID NO:2. In other embodiments, the animal comprises one allele of the gene that encodes a polypeptide having a sequence set forth in SEQ ID NO:2, and another allele of the gene that encodes a polypeptide having a sequence set forth in SEQ ID NO:3-19. In yet other embodiments, the animal comprises two alleles of the gene that each independently encodes a polypeptide having a sequence set forth in SEQ ID NO:3-19.

Also disclosed herein is a method of identifying a compound having therapeutic effect for HIBM, the method comprising administering the compound to a recombinant cell; and measuring the effect of the compound on a rate of production, or an extent of production, of sialic acid or CMP-sialic acid by the cell, where the recombinant cell comprising a nucleic acid sequence that encodes a polypeptide having at least 80% sequence identity to the sequence set forth in SEQ ID NO:2. In some embodiments, the recombinant cell comprises the vector, wherein the vector comprises a nucleic acid sequence that encodes a polypeptide having at least 80% sequence identity to the sequence set forth in SEQ ID NO:2. In some of these embodiments, the polypeptide has a sequence selected from the group consisting of set forth in SEQ ID NO:2 through SEQ ID NO:19. In some of these embodiments, the recombinant cell is in vitro. In other embodiments, the recombinant cell is a cell of a recombinant animal. In some of these embodiments, the recombinant cell is in vivo. In certain embodiments, the recombinant animal is made by the process of producing a vector comprising a nucleic acid sequence that encodes a polypeptide having at least 80% sequence identity to the sequence set forth in SEQ ID NO:2; producing a recombinant mammalian embryonic stem cell by infecting a mammalian embryonic stem cell with the vector; selecting the embryonic stem cell line that has undergone homologous recombination to incorporate the vector sequence at a desired genomic locus; planting the recombinant stem cell in an embryo; implanting the embryo in a female animal; and allowing the implanted embryo to mature into a fully formed fetus and be born from the female animal.

### BRIEF DESCRIPTION OF THE DRAWINGS

FIG. 1 is a scheme showing the resultant murine GNE (Uea1) genomic locus, exons 11 and 12, after homologous recombination with the targeting vector. The M712T missense mutation was created in exon 12 and a neo cassette (under the PGK promoter) was inserted, flanked by FRT sites. LoxP sites were inserted before exon 12 and after the PGK-neo gene.

FIG. 2 is a photograph of a gel showing the genotyping of mutant mice. PCR amplification of genomic DNA across the M712T (ATG>ACG) mutation site yielded a 387-bp fragment that was digested by the NlaIII restriction endonuclease into 354-bp and 33-bp fragments in a normal allele (+) and into 265-bp, 89-bp and 33-bp in the mutant allele (−).

FIG. 3 is a photograph of a gel showing the results of RT-PCR of kidney and skeletal muscle RNA. RNA was reverse transcribed onto cDNA and amplified by PCR with primers covering exons 11 and 12 (355-bp). Digestion by NlaIII cuts the normal allele (+) into 225-bp, 89-bp, and 41-bp fragments; the M712T allele (−) is cut into 314-bp and 41-bp fragments. Digestion confirmed the exclusive presence of the normal allele in +/+tissues, both alleles in +/−tissues, and only the M712T allele in −/−tissues.

FIG. 4 is a graph showing numbers of mice at embryonic age E17-19 and at weaning age P21. At P21, genotyping of 76 mice from 13 litters (9+/−matings) identified only one −/−offspring. Subsequent genotyping of 35 E17-19 embryos from 4 +/−matings yielded a Mendelian distribution of genotypes.

FIG. 5 is a table disclosing serum metabolite levels in weaned male mice, measured biweekly between age 6-8 months. BUN=Blood Urea Nitrogen.

### DETAILED DESCRIPTION OF THE EMBODIMENTS

In one aspect, disclosed herein is a method of treating HIBM in a subject comprising identifying subject in need thereof and administering to the subject a compound, or a pharmaceutically acceptable salt, ester, amide, glycol, peptidyl, or prodrug thereof, wherein the compound is a compound that is biosynthesized in a wild type individual along the pathway between glucose and sialic acid, inclusive.

The term “pharmaceutically acceptable salt” refers to a formulation of a compound that does not cause significant irritation to an organism to which it is administered and does not abrogate the biological activity and properties of the compound. Pharmaceutical salts can be obtained by reacting a compound disclosed herein with inorganic acids such as hydrochloric acid, hydrobromic acid, sulfuric acid, nitric acid, phosphoric acid, or organic acids such as methanesulfonic acid, ethanesulfonic acid, p-toluenesulfonic acid, salicylic acid and the like. Pharmaceutical salts can also be obtained by reacting a compound disclosed herein with a base to form a salt such as an ammonium salt, an alkali metal salt, such as a sodium or a potassium salt, an alkaline carth metal salt, such as a calcium or a magnesium salt, a salt of organic bases such as dicyclohexylamine, N-methyl-D-glucamine, tris(hydroxymethyl)methylamine, and salts with amino acids such as arginine, lysine and the like.

The term “ester” refers to a chemical moiety with formula —(R)ₙ—COOR′, where R and R′ are independently selected from the group consisting of alkyl, cycloalkyl, aryl, heteroaryl (bonded through a ring carbon) and heteroalicyclic (bonded through a ring carbon), and where n is 0 or 1.

An “amide” is a chemical moiety with formula —(R)ₙ—C(O)NHR′ or —(R)ₙ—NHC(O)R′, where R and R′ are independently selected from the group consisting of alkyl, cycloalkyl, aryl, heteroaryl (bonded through a ring carbon) and heteroalicyclic (bonded through a ring carbon), and where n is 0 or 1. An amide may be an amino acid or a peptide molecule attached to a compound disclosed herein, thereby forming a prodrug.

Any amine, hydroxy, or carboxyl side chain on the compounds disclosed herein can be esterified or amidified. The procedures and specific groups to be used to achieve this end is known to those of skill in the art and can readily be found in reference sources such as Greene and Wuts, Protective Groups in Organic Synthesis, 3.sup.rd Ed., John Wiley & Sons, New York, N.Y., 1999, which is incorporated herein in its entirety.

A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. An example, without limitation, of a prodrug would be a compound disclosed herein which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moicty.

In some embodiments, the compound is UDP-GlcNAc. In other embodiments, the compound is ManNAc. In yet other embodiments, the compound is ManNAc-6-P. In further embodiments, the compound is NeuAc-9-P. In some embodiments, the compound is sialic acid.

In some embodiments, provision of free sialic acid attenuates the hyposialylation in HIBM muscle and ameliorates the myopathic symptoms. In some of these embodiments, sialic acid is administered in its free form, bound as glycoconjugates, or as its precursor, ManNAc, which is uncharged and crosses membranes readily.

ManNAc is also situated in the sialic acid biosynthesis pathway after the rate-limiting UDP-GlcNAc 2-epimerase step, so its metabolism is not subject to feedback inhibition. In some embodiments, residual ManNAc kinase activity in HIBM patients, or ancillary kinases such as GlcNAc kinase, converts ManNAc into ManNAc-6P for subsequent synthesis of sialic acid. In fact, hyposialylated, GNE-deficient mouse embryonic stem cells became resialylated after their growth medium was supplemented with ManNAc [Schwarzkopf, 2002]. Furthermore, incubation of cultured cells with ‘unnatural’ ManNAc derivatives (ManLev, N-levulinoylmannosamine or ManNAz, N-azidoacetylmannosamine) resulted in incorporation of the downstream sialic acid analogs, SiaLev or SiaNAz, into cell surface glycoconjugates [Luchansky, 2003; Charter, 2002].

It is useful to test therapeutic methods involving the provision of sialic acid, or other therapeutic compounds, in animal models of HIBM. Animal models are developed by creating a knock-in animal model having a homozygous for the classic M712T mutation.

Thus, in another aspect, disclosed herein is a vector comprising a sequence set forth in SEQ ID NO:1. In some embodiments, the vector comprises a mutated gene. In some of these embodiments, the mutation is M712T mutation. By a gene having a, for example, M712T mutation it is meant that the gene encodes a polypeptide having the sequence set forth in SEQ ID NO:2, except that the methionine at position 712 is replaced with a threonine. In other embodiments, the mutation is selected from the group consisting of R8X, R71W, I142T, W204X, V216A, R246Q, I298T, R335W, Q436X, L556S, V572L, I587T, S615X, A631V, Y675H, and V696M.

In another aspect, disclosed herein is a vector comprising a gene, wherein the gene encodes a polypeptide having a sequence set forth in SEQ ID NO:2, or a polypeptide having at least 80% sequence identity to the sequence set forth in SEQ ID NO:2. In some embodiments, the encoded polypeptide has at least at least 85% sequence identity to the sequence set forth in SEQ ID NO:2. In other embodiments, the encoded polypeptide has 90% sequence identity to the sequence set forth in SEQ ID NO:2. In other embodiments, the encoded polypeptide has at least 95% sequence identity to the sequence set forth in SEQ ID NO:2. In other embodiments, the encoded polypeptide has at least 99% sequence identity to the sequence set forth in SEQ ID NO:2.

In some embodiments, the vector comprises a gene, wherein the gene encodes a polypeptide having a sequence set forth in SEQ ID NO:2, except that the polypeptide comprises a mutation selected from the group consisting of R8X (SEQ ID NO:3), R71W (SEQ ID NO:4), I142T (SEQ ID NO:5), W204X (SEQ ID NO:6), V216A (SEQ ID NO:7), R246Q (SEQ ID NO:8), I298T (SEQ ID NO:9), R335W (SEQ ID NO:10), Q436X (SEQ ID NO:11), L556S (SEQ ID NO:12), V572L (SEQ ID NO:13), I587T (SEQ ID NO:14), S615X (SEQ ID NO:15), A631V (SEQ ID NO:16), Y675H (SEQ ID NO:17), V696M (SEQ ID NO:18), and M712T ((SEQ ID NO:19). By a polypeptide having a, for example, M712T mutation it is meant that the polypeptide has the sequence set forth in SEQ ID NO:2, except that the methionine at position 712 is replaced with a threonine. The R8X mutation incorporates a stop codon. The resulting expressed polypeptide will only have the first seven amino acids of the SEQ ID NO:2.

In another aspect, disclosed herein is a recombinant cell comprising the vector described above. In some embodiments, the recombinant cell is a stem cell. In some of these embodiments, the recombinant cell is an embryonic stem cell. In some embodiments, the recombinant cell is a mammalian cell. In some embodiments, the recombinant cell is a mammalian stem cell, which can be a mammalian embryonic stem cell. In some embodiments, the mammal is selected from the group consisting of mouse, rat, rabbit, guinea pig, dog, cat, sheep, goat, cow, horse, monkey, chimpanzee, and ape. In some embodiments, the mammal is a primate. In other embodiments, the mammal is a murine.

In another aspect, disclosed herein is a recombinant animal comprising a recombinant cell described above. In some embodiments, the animal is a mammal. In some of these embodiments, the mammal is selected from the group consisting of mouse, rat, rabbit, guinea pig, dog, cat, sheep, goat, cow, horse, monkey, chimpanzee, and ape. In some embodiments, the mammal is a primate. In other embodiments, the mammal is a murine.

In another aspect, disclosed herein is a recombinant animal made by the process of:
producing a vector comprising a nucleic acid sequence that encodes a polypeptide selected from the group consisting of SEQ ID NO:2-19;
producing a recombinant mammalian embryonic stem cell by infecting a mammalian embryonic stem cell with the vector;
selecting the embryonic stem cell line that has undergone homologous recombination to incorporate the vector sequence at a desired genomic locus;
planting the recombinant stem cell in an embryo;
implanting the embryo in a female animal; and
allowing the implanted embryo to mature into a fully formed fetus and be born from the female animal.

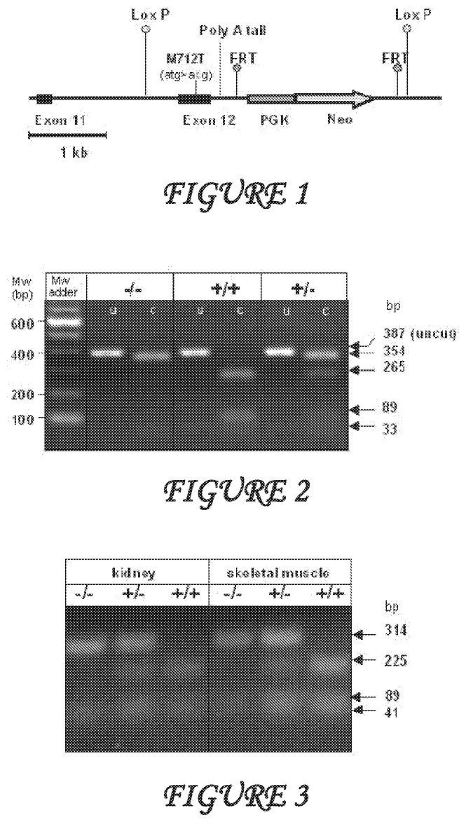

FIG. 1 is a schematic depiction of the genomic locus where the homologous recombination to incorporate the vector sequence is desired to undergo.

In some embodiments, the recombinant animal comprises one allele encoding for SEQ ID NO:2 and another allele encoding for a polypeptide selected from the group consisting of SEQ ID NO:3-19. In another embodiment, in the recombinant animal both alleles encode for a polypeptide selected from the group consisting of SEQ ID NO:3-19.

In another aspect, disclosed herein is a method of identifying a compound having therapeutic effect for Hereditary Inclusion Body Myopathy (HIBM), the method comprising: administering the compound to a recombinant cell; and measuring the effect of the compound on a rate of production, or an extent of production, of sialic acid or CMP-sialic acid by the cell, where the recombinant cell comprises a nucleic acid sequence that encodes a polypeptide having at least 80% sequence identity to the sequence set forth in SEQ ID NO:2.

In some embodiments, the recombinant cell comprises a vector, where the vector comprises a nucleic acid sequence that encodes a polypeptide having at least 80% sequence identity to the sequence set forth in SEQ ID NO:2. In some of these embodiments, the polypeptide has a sequence selected from the group consisting of set forth in SEQ ID NO:2 through SEQ ID NO:19.

In some embodiments, the recombinant cell is in vitro. In these embodiments, a particular cell is transformed and the cell is used in assays to identify therapeutic compounds without the cell being a part of a recombinant animal. The cell is, for example, used in standard and known cellular assays that detect the expression of polypeptides in cells in vitro.

In other embodiments, the recombinant cell is a cell of a recombinant animal. In some of these embodiments, the cell is part of a recombinant animal, or a tissue obtained from a recombinant animal. Thus, in these embodiments, the recombinant cell is in vivo. In some embodiments, the recombinant animal is made by the processes disclosed herein.

There are many well-known methods in the art to measure the therapeutic effect of a compound. In some ways, the same criterion is measured before and after the administration of the compound and the two measurements are compared. In other embodiments, a number of subjects are divided into at least two groups, where one receives the compound and the other receives a placebo, the same criterion is measured in the two groups, and the two measurements are compared. The criterion can be, for example, muscle movement, limb movement, muscle growth, muscle stamina, muscle fatigability, muscle strength, muscle tensile force, muscle atrophy, neuronal atrophy, life-span, extent of activity, and the like.

The terms “treating,” “treatment,” and “therapeutic” do not necessarily mean total cure. Any alleviation of any undesired signs or symptoms of the disease to any extent or the slowing down of the progress of the disease can be considered treatment. Furthermore, treatment may include acts that may worsen the aptient's overall feeling of well being or appearance. Treatment may also include lengthening the life of the subject, even if the symptoms are not alleviated, the disease conditions are not ameliorated, or the subject's overall feeling of well being is not improved.

In one particularly preferred embodiment, a gene-targeted knock-in mouse model homozygous for the classic M712T mutation was created. This mouse died within 72 hours after birth, at which time a muscle phenotype was not present. Instead, homozygous mice had severe glomerular disease, including fusion of the podocyte slit diaphragm membranes, possibly due to hyposialylation of specific membrane glycoproteins. Administration of ManNAc to pregnant mothers had a remarkably salutary effect on survival of homozygous pups and was associated with increased sialylation of PSA-NCAM as well as increased expression of GNE/MNK protein and its epimerase activity, suggesting that ManNAc might be stabilizing the mutant enzyme.

### EXAMPLES

The following examples are only illustrative of some of the embodiments of the invention disclosed herein and are not meant to limit the invention in any form.

### Example 1: Methods

GNE-M712T mice. GNE-M712T knock-in mice were generated by targeting the M712T (ATG>ACG) mutation exon 12 of the murine GNE gene (Uae1, Gne, GenBank NM_015828). The mutant mice were maintained in the C57BL/6J background. Animals were housed in ventilated cages in a temperature- and light-controlled environment (22° C., 30-70% humidity, 12-hour light/12-hour dark cycle) and were fed irradiated chow (Prolab 5P75 Isopro 3000; PMI Nutrition International) and water ad libitum. All euthanasia was performed with by CO₂ followed by cervical dislocations. For Mendelian distribution studies, four pregnant mice E17-19 were euthanized and embryos were retrieved by cesarean section and euthanized by decapitation. All mouse procedures were performed in accordance with protocol G04-3 approved by the Institutional Animal Core and Use Committee of the National Human Genome Research Institute, National Institutes of Health Institutional Review Board.

Molecular analysis. Mouse genotyping was performed on tail genomic DNA or cDNA isolated from murine kidney or skeletal muscle using standard protocols. Total RNA was isolated from murine tissues using the TRIzol reagent (Invitrogen Life Technologies), and cDNA was prepared using the SuperScript III system (Invitrogen Life Technologies). PCR amplifications were performed across the M712T mutation with genomic DNA as template, using the primerset 5′-AGCACTTCCTGGAGTTTGATG-3′ (SEQ ID NO:20) and 5′-ATTTGCCTTCGCAGAAACACTTGA-3′ (SEQ ID NO:21) or with cDNA as template using the primerset 5′-GCCCAGAGCATCTTACGAAC-3′ (SEQ ID NO:22) and 5′-GGGTCCCCTGGAGCTTGG-3′ (SEQ ID NO:23) and PuReTaq Ready-To-Go PCR beads (Amersham Biosciences), using standard PCR conditions. PCR fragments were digested with Nla III at 37° C. to verify the mutation status. Quantitative real-time PCR was performed on RNA isolated from kidney and skeletal muscle, utilizing assays-on-demand (Applied Biosystems) for GNE (mm00607939_s1) and β-actin (mm00450174_m1) on an ABI PRISM 7900 HT Sequence Detection System (Applied Biosystems).

Clinical Chemistry Screen. Blood samples (100-150 μl) from weaned, sex (male) and age (starting at age 6 months) matched mice (weighing at least 15 grams) were obtained bi-monthly, by puncture of anesthetized retro-orbital sinus (0.5% tetracaine HCl, Bausch and Lomb Pharmaceuticals). Samples were allowed to clot (30 min, room temperature) in MicroPrep centrifuge tubes (StatSpin), after which the serum was separated by centrifugation at 1500 g for 10 min, and stored at −80° C. until analysis. Clinical Chemistry screens were performed at the Department of Laboratory Medicine at the National Institutes of Health (http://www.cc.nih.gov/cp/index.shtml) and included monitoring of creatinine, blood urca nitrogen (BUN), albumin, total protein, uric acid, alkaline phosphatase, alanine aminotransfercase (ALT), aspartate aminotransferase (AST), amylase, creatine kinase and lactate dehydrogenase. In addition, reagent strips for protein urinalysis were used to assess proteinuria in urine from mice (Chemstrip 2GP; Roche).

Antibodies. A rabbit polyclonal antibody was custom prepared against a GNE/MNK peptide comprising amino acids 588-607: EAYASGMALQREAKKLHDED (SEQ ID NO:24), coupled to keyhole limpet hemocyanine (KLH) and affinity-purified against the corresponding antigenic peptide (Covance). Other primary antibodies were commercially obtained: dystrophin (ab15277, AbCam), α-dystroglycan (IIH6, Upstate Biotechnology), laminin (L9393, Sigma-Aldrich), podocalyxin (PODX15-A, Alpha Diagnostic International), PSA-NCAM (MAB5324, Chemicon), and β-actin (AAN01, Cytoskeleton).

Mouse Histology. Mouse tissues were collected, formalin-fixed and paraffin-embedded. Tissue sections (5 μm) were prepared and stained with hematoxylin and cosin following standard procedures (American Histolabs) or subjected to immunohostochemistry with a variety of primary antibodies. Formalin fixed tissues were deparafinized in Histoclear II (National Diagnostics), dehydrated in a series of ethanol solutions. Antigen retrieval was performed for sections to be stained with the antibodies GNE/MNK (5 min boiling in citric acid based solution; Vector Laboratories) and dystrophin (boiling in 1 mM EDTA, according the manufacturer's protocol; AbCam). The sections were blocked (2% BSA, 10% donkey scrum and 0.1% Triton X-100 in PBS) and incubated with primary antibodies (GNE/MNK 1:50; laminin 1:25; dystrophin 1:50) overnight at 4° C., followed by the secondary antibody Alexa 488 donkey-anti-rabbit (1:500 in blocking solution) (Invitrogen). The sections were mounted in Vectashield (Vector Laboratories) and viewed and digitally imaged with a Zeiss Axiovert 200M microscope (Carl Zeiss, Microimaging).

Western blotting. Mouse tissues (age P2) were extracted, homogenized in CelLytic buffer, consisting of a mild detergent, bicine buffer and 150 mM NaC (CelLytic) supplemented with protease inhibitors (Complete Miniâ, Roche). The lysates were sonicated and cleared by centrifugation (1000 g for 10 min), and the resulting supernatants were assayed for protein concentration (BCA protein assay, Pierce). For the neuraminidase enzymatic treatments, protein homogenates (25 μg) were incubated with 1 mU/μg of Neuraminidase (N-6514, Sigma) for 30 min at 37° C. Equal amounts of protein (25-50 μg) were electrophoresed on 4-12% Tris-Glycine gels (Novex, Invitrogen), and electroblotted onto 0.45 μm Hybond ECL nitrocellulose membranes (Amersham Pharmacia Biotech). The membranes were blocked (10% fat-free milk) and incubated with primary antibodies, followed by incubation with HRP-conjugated secondary antibodies (Amersham Biosciences). Results were visualized with enhanced chemiluminescence (ECL Western Blotting Detection Reagents, Amersham Biosciences) and exposure to CL-XPosure film (Pierce Biotechnology). Densitometry was performed on the digital images obtained with a Kodak Image station and software (Perkin Elmer). The protein levels were normalized to those of B-actin to correct for differences in protein loading and/or transfer.

Electron Microscopy. Kidney tissues were fixed overnight at 4° C. in 2% glutaraldehyde in 0.1M cacodylate buffer (pH 7.4), followed by washing with cacodylate buffer and postfixation with 1% OsO4 for 2 h. After washing (0.1 M cacodylate buffer), the tissues were serially dehydrated in ethanol and embedded in Eponate 12 resin (Ted Pella). Thin sections (˜80 nm), were obtained by a Leica ultracut-UCT ultramicrotome (Leica), placed onto 400 mesh copper grids, and stained with saturated uranyl acetate in 50% methanol, followed by lead citrate. The grids were viewed with a Philips 410 electron microscope (FEI Company) at 80 kV and images were recorded on Kodak SO-163 film (Kodak).

ManNAc administration. Breeding pairs of 6-week-old+/−mice were divided into three groups. Group I consisted of 9+/−breeding pairs who were administered untreated sterilized tap water. Group II consisted of one breeding pair of +/+mice (wild-type control) and 6+/−breeding pairs who were administered 1 mg/ml ManNAc (Sigma) supplemented water. Group III consisted of one +/+breeding pair and 7+/−breeding pairs who were administered 5 mg/ml ManNAc supplemented water. Water was changed twice weekly. Nursing females continued to be supplied with ManNAc. All mice were weaned from ManNAc at 21 days. Selected whole litters were sacrificed at age P2 or P6 for histological, genetic, biochemical or ultrastructural analysis.

GNE/MNK enzymatic assays. Mouse kidney and skeletal muscle (hindlimb quadriceps) tissues were homogenized and subjected to the GNE/MNK epimerase enzymatic assay as described^{11,44}. This assay is based on incubation with radiolabeled substrate (UDP-[³H]GlcNAc; American Radiolabeled Chemicals), and detection of radiolabeled product ([³H]ManNAc) upon separation of oligosaccharides by high pH anion-exchange chromatography with pulsed amperometric detection (Dionex).

Statistical analysis. Differences in genotype distribution (+/+:+/−:−/−) between an untreated control group and ManNAc treated group were tested by a two-tailed Fisher's exact test. To this end a 2×3 table was generated.

### Example 2: Generation and Examination of GNE-M712T Knock-In Mice

A murine targeting vector for homologous recombination in C57BL/6J embryonic stem cells was constructed including the M712T GNE mutation (FIG. 1). The neo (neomycin phosphotransferase) and tk (thymidine kinase) genes were introduced into the vector as positive and negative selection markers, respectively (FIG. 1). Additional LoxP (flanking exon 12 and neo) and FRT (flippase recombinase target) sites (flanking neo) were inserted for potential future conditional transgenic models. Genotyping of the mice was performed by PCR amplification and digestion with the restriction endonuclease NlaIII (FIG. 2). Tissues of homozygous mutated (−/−) mice and wild type mice (+/+) showed comparable GNE RNA transcript levels by real-time PCR (not shown). Furthermore, NlaIII digestion of amplified cDNA demonstrated homozygous insertion of the M712T mutation in RNA of −/−mice (FIG. 3).

Early postnatal lethality of −/−mice

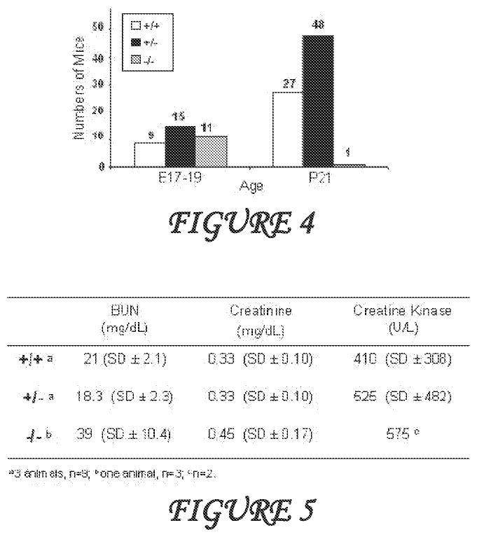

Initial matings of heterozygous (+/−) mice yielded only one −/−animal at weaning age (postnatal day 21; P21). However, subsequent genotyping of 35 embryos at day E17-19 showed 26% (+/+): 43% (+/−): 31% (−/−) in utero, equaling a Mendelian distribution, statistically confirmed by goodness of fit testing (x²=0.94, P=0.62) (FIG. 4). At E17-19, the embryos displayed normal exteriors, normal head and body sizes, and normal pink skin, indicating good circulatory and respiratory function. By P2, however, −/−mice appeared slightly smaller, with weights 70-100% of those of control littermates. The −/−mice stomachs contained milk upon dissection, although a milkspot was not always visible. By P3, all −/−mice except one had died. In contrast, +/−mice appeared entirely unaffected.

Serum metabolite analyses of weaned mice revealed elevated blood urea nitrogen (BUN) levels and excessive amounts of urinary protein for the −/−mice, indications of renal disease (FIG. 5). Blood creatinine levels were within the normal range in all genotypes and creatine kinase levels were normal to slightly elevated in −/−mice (FIG. 5). All other serum metabolites tested were within the normal range.

### Histological Analyses of Knock-In Mice

Tissues of −/−mice and their littermates were examined histologically between age P2 and P3. Particular attention was paid to skeletal muscle, heart and liver; no abnormalities were identified in these tissues. Moreover, immuno-histochemical staining with antibodies against laminin and dystrophin failed to show differences between −/−and +/+muscle sections. The monoclonal antibodies against glycosylated α-dystroglycan and against PSA-NCAM¹⁵ were raised in mouse, and failed to give satisfactory histological staining results.

At age P2, kidneys of −/−mice showed hemorrhages by gross examination, but were normal in size and shape compared to kidneys of +/+and +/−littermates. Histological analyses revealed cystic dilatations in the cortex and medulla. Higher magnification views of −/−kidneys displayed collecting ducts, proximal and distal convoluted tubules, and urinary space filled with red cells and fibrillar infiltrates, indicating that blood had leaked into the tubules. The glomeruli of −/−mice contained red cell infiltrations in Bowman's space. Quantitatition of affected glomeruli in 4 mice (total of ˜100 glomeruli scored) yielded 64 (±6)% affected in −/−mice versus 2 (±1) % affected in +/−and 4 (±3.5)% in +/+micc. Immunohistochemistry with anti-GNE/MNK antibodies demonstrated localization of GNE/MNK protein to kidney glomeruli. Examinations of −/−kidneys at E18 showed no histological differences, nor developmental delay compared with normal or heterozygous littermates (not shown). Other renal disorders also become apparent only after birth, when the mice transfer from maternal to independent renal filtering of blood.

The only homozygous −/−survivor past weaning (P21) was smaller than his +/+and +/−littermates, but he continued to grow and gain weight until about 7 months, after which he failed to grow and was sacrificed at age 8.5 months, along with two +/+and +/−littermates. Necropsy data revealed the tubules and glomeruli contained red cell infiltrates, likely due to lesions in their epithelial linings. These renal abnormalities account for the elevated BUN levels in this mouse (FIG. 5). Detailed investigations of other tissues, in particular hindlimb and forclimb musculature, did not yield any abnormalities (structural or inflammatory). Ultrastructural analysis of −/−glomeruli revealed that the podocyte foot-process membranes were flattened and largely fused, with only a few, wide foot processes present. Filtration slits were largely reduced in number and showed formation of tight junctions. The size and shape of the glomerular basement membrane (GMB) seemed intact, with a few areas where it was reduced in size. Endothelial cells lining the basement membrane did not show ultrastructural defects. Red cells were found infiltrated in kidney tubules and were frequently odd-shaped, likely due to osmotic and/or pH environmental changes.

### Rescue of the Knock-In Mice by Mannac Feeding

ManNAc, added to the drinking water at a concentration of 1 mg/ml during matings of +/−mice did not yield any surviving homozygous mice beyond age P3 from among 51 offspring. However, at 5 mg ManNAc/ml, 11 homozygous (44% of total −/−pups) out of a total of 97 newborn pups survived beyond P3. The nursing females continued to be supplied with ManNAc until the pups were weaned (P21). Of the 11−/−survivors past P3, 7 died after between P6 and P12, and another 2 were missing at P9. Two homozygous mice survived past P21, when ManNAc supplementation ceased. These 2 mice were smaller than their littermates, but continued to grow without receiving additional ManNAc. At 3.5 months old, one −/−survivor was sacrificed due to a debilitated physical condition. H&E histology on skeletal muscle of this mouse did not reveal any structural or inflammatory abnormalities, but the kidneys showed mild disease, including red cell infiltration in glomeruli and fibrillar inclusions in the tubules. The one surviving −/−mouse is currently 6 months old and does not exhibit apparent myopatic features.

The ManNAc treated −/−mice (and littermates) were sacrificed at age P6 to assess their tissue histology. At this age, homozygous mutated mice that could not survive past P3 were already eliminated and mice that would possibly die before weaning (age P21) were included. No abnormalities in liver, heart and skeletal muscle tissues were identified in the P6, −/−, ManNAc treated mice (not shown). Detailed histological investigations of their kidneys demonstrated a range from mildly to remarkably improved histological features. After ManNAc treatment, the number of cystic dilatations in the cortex and medulla were reduced as well as the degree of red cell infiltrates in glomeruli, in the tubular and in the urinary space. Ultrastructurally, improvement was noticeable in the severity of the fusion and flattening of the podocyte foot processes, including higher numbers of slit diaphragms and more ‘finger-shaped’ foot processes.

### Biochemical Analyses After ManNAc Feeding

UDP-GlcNAC 2-epimerase (GNE) enzymatic activity in muscle and kidney at age P2 were determined. Compared to +/+mice, which were set at 100% (n=4) activity, −/−mice muscle was reduced to 19 (+7)% GNE activity (n=4). Similar decreased GNE activities were measured in −/−kidney extracts (10% of normal, n=2). Upon ManNAc treatment, GNE activities in +/+muscle increased to 114 (±10)% (n=3), while −/−muscle increased to 31 (±9)% (n=7) residual activity.

Immunoblots of muscle and kidney extracts labeled with anti-GNE/MNK antibodies demonstrated 78 (±5)% decreased amounts of GNE/MNK protein in −/−tissues, which improved in ManNAc treated muscle and kidney to 41 (±3)% decreased amounts compared to +/+littermates (referenced to β-actin). PSA-NCAM antibodies showed a significantly increased signal of 2-28% (n=14 pre treatment and n=10 post treatment, p=0.08) in −/−brain tissues when compared to untreated −/−tissues. Staining patterns with antibodies against laminin, an integral component of the glomerular basement membrane²⁵, did not differ in laminin concentration or size in −/−mice kidney extracts, but antibodies against podocalyxin, the major sialoglycoprotein of the podocyte filtration slits, showed dramatically decreased sialylation in −/−kidneys. ManNAc feeding did not result in a statistically significant increase in podocalyxin sialylation status.

### Example 3: Discussion

GNE gene-targeted knock-in mice was created mimicking the M712T mutation of Persian-Jewish HIBM patients. Homozygous mutated mice progressed through embryonic life at a frequency predicted by Mendelian genetics, but, except for one anomalous male, did not survive past age P3. Histological studies on −/−mice showed no muscle pathology at age P2, but skeletal muscle and kidney GNE/MNK epimerase activity was one-fifth that of their normal littermates. These decreases could partly be due to reduced presence of GNE/MNK protein in −/−tissues.

At P2, kidney histology of −/−mice revealed glomerular disease, hemorrhages, and red cells and fibrillar infiltrates in renal tubules, glomeruli, urinary space, and Bowman's space. Electron micrographs of −/−glomeruli showed dramatically flattened and fused foot processes of podocytes with severely affected filter slits. This kidney involvement was unexpected, but pointed to the critical role of sialic acid in renal tissue. On immunohistochemistry, GNE/MNK localized to kidney glomeruli, where sialic acid is abundantly present. These large amounts of sialic acid may support the extensive sialylation of glycoproteins (such as α-dystroglycan, α3β1-integrin and podocalyxin) essential for the function of podocyte foot processes. Podocytes are renal glomerular epithelial cells that provide the architecture of the glomerular filtration apparatus, including interdigitating foot processes, slit diaphragms, and the intercellular urinary spaces. The negatively charged sialic acid residues on glycoproteins act as anti-adhesion molecules, assisting in maintaining an open urinary space, filtration slits and Bowman's space. Some forms of glomerular disease (such as minimal change nephrosis) can result from hyposialylation and subsequent deformation of podocyte membranes and the onset of proteinuria. Indeed, we demonstrated hyposialylated podocalyxin in −/−mice kidney extracts and electron micrographs showed flattening and fused foot processes lining the GBM, resulting in reduced numbers and malformed (forming tight junctions) filtration slits. The GBM appeared unaffected but displayed occasional thinner areas, which could be locations where red cells gained access to Bowman's space, as is seen in thin membrane disease or Alport syndrome. These severe renal findings in our −/−mice likely led to dehydration, other uremic complications, and death before age P3. Unexpectedly, this animal model might provide an opportunity to study basic mechanisms and targeted therapies of podocyte injuries, for which appropriate model systems are sparse.

Our murine findings were in contrast to the exclusively myopathic involvement in human HIBM. The carly death of −/−mice did not allow for studying a possible muscular phenotype at an older age, but at P2, hyposialylation of α-dystroglycan, reported in human HIBM muscle, was not observed in −/−mouse muscle. There are several possible reasons for this. Glycosylation patterns of α-dystroglycan are complex and tissue-specific, and might also be species-specific. Renal physiology and kidney sialic acid metabolism might also differ between humans and mice. The kidney harbors specialized biosynthetic machinery for protein polysialylation, and the type of sialic acid present in human and mouse kidneys might differ. Most mammalian species utilize the sialic acid Neu5Gc (N-glycolylneuraminic acid), rather than Neu5Ac. However, humans have lost the ability to synthesize NeuGe, and rely on Neu5Ac as their main sialic acid. Mice not only have relatively little Neu5Ac, they also have negligible ability to deaminate Neu5Ac to form KDN (2-keto-3-deoxy-D-glycero-D-galacto-nonulosonic acid). In kidney, KDN predominantly decorates O-linked glycans on megalin, a membrane glycoprotein abundant in the renal proximal tubule. It is also possible that the C57BL/6 genetic background of our murine model has a high susceptibility for renal defects. Future studies, such as outbreeding our mice to a different genetic background or employing the Cre-Lox system to create conditional GNE knock-outs, might shed light on these issues.

The −/−mice did exhibit an adverse biochemical effect of decreased sialic acid production, albeit not in kidney or muscle tissue. Immunoblotting of brain extracts showed hyposialylation of PSA-NCAM in the majority of the −/−mice. Hyposyialylation of PSA-NCAM was previously reported in embryonic stem cells of complete GNE knock-out mice, as well as in skeletal muscle tissue of HIBM patients.

Whatever the reason for the carly death of the M712T homozygous mice, we were able to rescue this phenotype by administering ManNAc (1 g/kg/day) to +/−breeding pairs. In fact, 42% of the −/−offspring survived past P3 and 18% of these survivors lived past weaning age P21. Histological studies at age P6 showed mildly to significantly improved kidney histology as well as increased sialylation on brain PSA-NCAM. Two mice surviving past P21, no longer supplied with ManNAc, were smaller and lighter than their littermates, but continued to grow.

Remarkably, ManNAc supplementation increased skeletal muscle GNE/MNK epimerase activity from 100% to ˜114% in +/+mice, and from 19% to 31% in −/−mice. In addition, immunoblotting revealed an increased amount of GNE/MNK protein subsequent to ManNAc feeding. These findings suggest that ManNAc might stabilize both the normal and the mutant enzymes, increasing catalytic activity. Similar stabilization effects on other proteins have been demonstrated using natural or artificial ligands or chaperones. The effects of ManNAc on GNE mutations other than M712T, are worth future investigations.

The M712T GNE knock-in mouse, despite its lack of early myopathic features, nevertheless provides a suitable model for human HIBM. First, the human disease also lacks carly muscle impairment, and the M712T mouse may prove to display HIBM-like myopathy if it can be maintained well past weaning. Second, survival of the M712T mouse past P3 can serve as an absolute outcome parameter for potential therapeutic interventions, and resolution of renal disease provides a graded measure of response.

In fact, both of these outcome measures indicated a significant salutary effect of ManNAc supplementation in the M712T mouse. Although the exact mechanism of ManNAc's beneficial effect has not been proven, the apparent stabilization of UDP-GlcNAc 2-epimerase activity, combined with the known existence of ancillary enzymes providing ManNAc kinase activity, suggests that increased ManNAc supplementation is effecting increased sialic acid production. Indeed, there appears to be more sialylated PSA-NCAM in the brains of ManNAc-treated compared with untreated knock-in mice. These findings support the hypothesis that the provision of sialic acid may improve the myopathy of HIBM. Indeed, preliminary evidence indicates a mild, transient, but significant improvement in the muscle strength of HIBM patients who received intravenous immune globulin G, and it is hypothesized that this effect is mediated through the large sialic acid content provided by the immune globulin. We think that the uncharged, physiological monosaccharide ManNAc is a promising candidate-drug for a clinical trial in patients with HIBM, in particular those patients harboring the M712T GNE mutation. Other sialic acid precursors may also be reasonable candidates, especially if they show efficacy in the appropriate mouse model of HIBM.

### References

The following references are incorporated by reference herein in their entirety:
1. Schwarzkopf, M. et al. Sialylation is essential for early development in mice. Proc. Natl. Acad. Sci. U. S. A. 99, 5267-5270 (2002).
2. Luchansky, S. J., Yarema, K. J., Takahashi, S. & Bertozzi, C.R. GlcNAc 2-epimerase can serve a catabolic role in sialic acid metabolism. J. Biol. Chem. 278, 8035-8042 (2003).
3. Charter, N. W., Koshland, D. E. Jr. & Bertozzi, C. R. Biosynthetic incorporation of unnatural sialic acids into polysialic acid on neural cells. Glycobiology 10, 1049-1056 (2000).

1-20. (canceled)

21. A method of treating myopathy in a subject harboring one or more mutations in the gene encoding a bifunctional UDP-N-acetylglucosamine 2-epimerase/N-acetylmannosamine kinase (GNE), comprising orally administering to the subject a composition comprising an effective amount of sialic acid or a pharmaceutically acceptable salt or prodrug thereof.

22. The method of claim 21, wherein the subject harbors a M712T mutation in GNE.

23. The method of claim 21, wherein subsequent to the administration of the composition, the subject experiences an improvement in a criterion selected from the group consisting of muscle movement, limb movement, muscle growth, muscle stamina, muscle fatigability, muscle strength, muscle tensile force, muscle atrophy, neuronal atrophy, life-span, and extent of activity.

24. The method of claim 21, wherein administration of the composition results in enhanced muscle strength in the subject compared to a subject receiving placebo.

25. The method of claim 21, wherein administration of the composition results in enhanced limb movement in the subject compared to a subject receiving placebo.

26. The method of claim 21, wherein the subject is identified as harboring a mutation in the gene encoding GNE prior to administration of the composition.

27. A method of treating myopathy in a subject, wherein the method comprises:
identifying one or more mutations in the gene encoding a bifunctional UDP-N-acetylglucosamine 2-epimerase/N-acetylmannosamine kinase (GNE) in a subject; and
orally administering to the subject a composition comprising an effective amount of sialic acid or a pharmaceutically acceptable salt or prodrug thereof.

28. The method of claim 27, wherein the subject is identified to harbor a M712T mutation in GNE.

29. The method of claim 27, wherein subsequent to the administration of the composition, the subject experiences an improvement in a criterion selected from the group consisting of muscle movement, limb movement, muscle growth, muscle stamina, muscle fatigability, muscle strength, muscle tensile force, muscle atrophy, neuronal atrophy, life-span, and extent of activity.

30. The method of claim 27, wherein administration of the composition results in enhanced muscle strength in the subject compared to a subject receiving placebo.

31. The method of claim 27, wherein administration of the composition results in enhanced limb movement in the subject compared to a subject receiving placebo.


SEQUENCE LISTING (24 sequences)

SEQ ID NO: 1 - DNA, 2169 bp, Homo sapiens
  source 1..2169: mol_type = other DNA
atggagaagaatggaaataaccgaaagctgcgggtttgtgttgctacttgtaaccgtgcagattattctaaacttgccccgatcatgtttggcattaaaaccgaacctgagttctttgaacttgatgttgtggtacttggctctcacctgatagatgactatggaaatacatatcgaatgattgaacaagatgactttgacattaacaccaggctacacacaattgtgaggggagaagatgaggcagccatggtggagtcagtaggcctggccctagtgaagctgccagatgtccttaatcgcctgaagcctgatatcatgattgttcatggagacaggtttgatgccctggctctggccacatctgctgccttgatgaacatccgaatccttcacattgaaggtggggaagtcagtgggaccattgatgactctatcagacatgccataacaaaactggctcattatcatgtgtgctgcacccgcagtgcagagcagcacctgatatccatgtgtgaggaccatgatcgcatccttttggcaggctgcccttcctatgacaaacttctctcagccaagaacaaagactacatgagcatcattcgcatgtggctaggtgatgatgtaaaatctaaagattacattgttgcactacagcaccctgtgaccactgacattaagcattccataaaaatgtttgaattaacattggatgcacttatctcatttaacaagcggaccctagtcctgtttccaaatattgacgcagggagcaaagagatggttcgagtgatgcggaagaagggcattgagcatcatcccaactttcgtgcagttaaacacgtcccatttgaccagtttatacagttggttgcccatgctggctgtatgattgggaacagcagctgtggggttcgagaagttggagcttttggaacacctgtgatcaacctgggaacacgtcagattggaagagaaacaggggagaatgttcttcatgtccgggatgctgacacccaagacaaaatattgcaagcactgcaccttcagtttggtaaacagtacccttgttcaaagatatatggggatggaaatgctgttccaaggattttgaagtttctcaaatctatcgatcttcaagagccactgcaaaagaaattctgctttcctcctgtgaaggagaatatctctcaagatattgaccatattcttgaaactctaagtgccttggccgttgatcttggcgggacgaacctccgagttgcaatagtcagcatgaagggtgaaatagttaagaagtatactcagttcaatcctaaaacctatgaagagaggattaatttaatcctacagatgtgtgtggaagctgcagcagaagctgtaaaactgaactgcagaattttgggagtaggcatttccacaggtggccgtgtaaatcctcgggaaggaattgtgctgcattcaaccaaactgatccaagagtggaactctgtggaccttaggacccccctttctgacactttgcatctccctgtgtgggtagacaatgatggcaactgtgctgccctggcggaaaggaaatttggccaaggaaagggactggaaaactttgttacacttatcacaggcacaggaatcggtggtggaattatccatcagcatgaattgatccacggaagctccttctgtgctgcagaactgggccaccttgttgtgtctctggatgggcctgattgttcctgtggaagccatgggtgcattgaagcatacgcctctggaatggccttgcagagggaggcaaaaaagctccatgatgaggacctgctcttggtggaagggatgtcagtgccaaaagatgaggctgtgggtgcgctccatctcatccaagctgcgaaacttggcaatgcgaaggcccagagcatcctaagaacagctggaacagctttgggtcttggggttgtgaacatcctccataccatgaatccctcccttgtgatcctctccggagtcctggccagtcactatatccacattgtcaaagacgtcattcgccagcaggccttgtcctccgtgcaggacgtggatgtggtggtttcggatttggttgaccccgccctgctgggtgctgccagcatggttctggactacacaacacgcaggatctactag

SEQ ID NO: 2 - protein, 722 aa, Homo sapiens
MEKNGNNRKLRVCVATCNRADYSKLAPIMFGIKTEPEFFELDVVVLGSHLIDDYGNTYRMIEQDDFDINTRLHTIVRGEDEAAMVESVGLALVKLPDVLNRLKPDIMIVHGDRFDALALATSAALMNIRILHIEGGEVSGTIDDSIRHAITKLAHYHVCCTRSAEQHLISMCEDHDRILLAGCPSYDKLLSAKNKDYMSIIRMWLGDDVKSKDYIVALQHPVTTDIKHSIKMFELTLDALISFNKRTLVLFPNIDAGSKEMVRVMRKKGIEHHPNFRAVKHVPFDQFIQLVAHAGCMIGNSSCGVREVGAFGTPVINLGTRQIGRETGENVLHVRDADTQDKILQALHLQFGKQYPCSKIYGDGNAVPRILKFLKSIDLQEPLQKKFCFPPVKENISQDIDHILETLSALAVDLGGTNLRVAIVSMKGEIVKKYTQFNPKTYEERINLILQMCVEAAAEAVKLNCRILGVGISTGGRVNPREGIVLHSTKLIQEWNSVDLRTPLSDTLHLPVWVDNDGNCAALAERKFGQGKGLENFVTLITGTGIGGGIIHQHELIHGSSFCAAELGHLVVSLDGPDCSCGSHGCIEAYASGMALQREAKKLHDEDLLLVEGMSVPKDEAVGALHLIQAAKLGNAKAQSILRTAGTALGLGVVNILHTMNPSLVILSGVLASHYIHIVKDVIRQQALSSVQDVDVVVSDLVDPALLGAASMVLDYTTRRIY

SEQ ID NO: 3 - protein, 7 aa, Homo sapiens
MEKNGNN

SEQ ID NO: 4 - protein, 722 aa, Homo sapiens
MEKNGNNRKLRVCVATCNRADYSKLAPIMFGIKTEPEFFELDVVVLGSHLIDDYGNTYRMIEQDDFDINTWLHTIVRGEDEAAMVESVGLALVKLPDVLNRLKPDIMIVHGDRFDALALATSAALMNIRILHIEGGEVSGTIDDSIRHAITKLAHYHVCCTRSAEQHLISMCEDHDRILLAGCPSYDKLLSAKNKDYMSIIRMWLGDDVKSKDYIVALQHPVTTDIKHSIKMFELTLDALISFNKRTLVLFPNIDAGSKEMVRVMRKKGIEHHPNFRAVKHVPFDQFIQLVAHAGCMIGNSSCGVREVGAFGTPVINLGTRQIGRETGENVLHVRDADTQDKILQALHLQFGKQYPCSKIYGDGNAVPRILKFLKSIDLQEPLQKKFCFPPVKENISQDIDHILETLSALAVDLGGTNLRVAIVSMKGEIVKKYTQFNPKTYEERINLILQMCVEAAAEAVKLNCRILGVGISTGGRVNPREGIVLHSTKLIQEWNSVDLRTPLSDTLHLPVWVDNDGNCAALAERKFGQGKGLENFVTLITGTGIGGGIIHQHELIHGSSFCAAELGHLVVSLDGPDCSCGSHGCIEAYASGMALQREAKKLHDEDLLLVEGMSVPKDEAVGALHLIQAAKLGNAKAQSILRTAGTALGLGVVNILHTMNPSLVILSGVLASHYIHIVKDVIRQQALSSVQDVDVVVSDLVDPALLGAASMVLDYTTRRIY

SEQ ID NO: 5 - protein, 722 aa, Homo sapiens
MEKNGNNRKLRVCVATCNRADYSKLAPIMFGIKTEPEFFELDVVVLGSHLIDDYGNTYRMIEQDDFDINTRLHTIVRGEDEAAMVESVGLALVKLPDVLNRLKPDIMIVHGDRFDALALATSAALMNIRILHIEGGEVSGTTDDSIRHAITKLAHYHVCCTRSAEQHLISMCEDHDRILLAGCPSYDKLLSAKNKDYMSIIRMWLGDDVKSKDYIVALQHPVTTDIKHSIKMFELTLDALISFNKRTLVLFPNIDAGSKEMVRVMRKKGIEHHPNFRAVKHVPFDQFIQLVAHAGCMIGNSSCGVREVGAFGTPVINLGTRQIGRETGENVLHVRDADTQDKILQALHLQFGKQYPCSKIYGDGNAVPRILKFLKSIDLQEPLQKKFCFPPVKENISQDIDHILETLSALAVDLGGTNLRVAIVSMKGEIVKKYTQFNPKTYEERINLILQMCVEAAAEAVKLNCRILGVGISTGGRVNPREGIVLHSTKLIQEWNSVDLRTPLSDTLHLPVWVDNDGNCAALAERKFGQGKGLENFVTLITGTGIGGGIIHQHELIHGSSFCAAELGHLVVSLDGPDCSCGSHGCIEAYASGMALQREAKKLHDEDLLLVEGMSVPKDEAVGALHLIQAAKLGNAKAQSILRTAGTALGLGVVNILHTMNPSLVILSGVLASHYIHIVKDVIRQQALSSVQDVDVVVSDLVDPALLGAASMVLDYTTRRIY

SEQ ID NO: 6 - protein, 722 aa, Homo sapiens
  VARIANT 204: note = X can be any naturally occurring amino acid
MEKNGNNRKLRVCVATCNRADYSKLAPIMFGIKTEPEFFELDVVVLGSHLIDDYGNTYRMIEQDDFDINTRLHTIVRGEDEAAMVESVGLALVKLPDVLNRLKPDIMIVHGDRFDALALATSAALMNIRILHIEGGEVSGTIDDSIRHAITKLAHYHVCCTRSAEQHLISMCEDHDRILLAGCPSYDKLLSAKNKDYMSIIRMXLGDDVKSKDYIVALQHPVTTDIKHSIKMFELTLDALISFNKRTLVLFPNIDAGSKEMVRVMRKKGIEHHPNFRAVKHVPFDQFIQLVAHAGCMIGNSSCGVREVGAFGTPVINLGTRQIGRETGENVLHVRDADTQDKILQALHLQFGKQYPCSKIYGDGNAVPRILKFLKSIDLQEPLQKKFCFPPVKENISQDIDHILETLSALAVDLGGTNLRVAIVSMKGEIVKKYTQFNPKTYEERINLILQMCVEAAAEAVKLNCRILGVGISTGGRVNPREGIVLHSTKLIQEWNSVDLRTPLSDTLHLPVWVDNDGNCAALAERKFGQGKGLENFVTLITGTGIGGGIIHQHELIHGSSFCAAELGHLVVSLDGPDCSCGSHGCIEAYASGMALQREAKKLHDEDLLLVEGMSVPKDEAVGALHLIQAAKLGNAKAQSILRTAGTALGLGVVNILHTMNPSLVILSGVLASHYIHIVKDVIRQQALSSVQDVDVVVSDLVDPALLGAASMVLDYTTRRIY

SEQ ID NO: 7 - protein, 722 aa, Homo sapiens
MEKNGNNRKLRVCVATCNRADYSKLAPIMFGIKTEPEFFELDVVVLGSHLIDDYGNTYRMIEQDDFDINTRLHTIVRGEDEAAMVESVGLALVKLPDVLNRLKPDIMIVHGDRFDALALATSAALMNIRILHIEGGEVSGTIDDSIRHAITKLAHYHVCCTRSAEQHLISMCEDHDRILLAGCPSYDKLLSAKNKDYMSIIRMWLGDDVKSKDYIAALQHPVTTDIKHSIKMFELTLDALISFNKRTLVLFPNIDAGSKEMVRVMRKKGIEHHPNFRAVKHVPFDQFIQLVAHAGCMIGNSSCGVREVGAFGTPVINLGTRQIGRETGENVLHVRDADTQDKILQALHLQFGKQYPCSKIYGDGNAVPRILKFLKSIDLQEPLQKKFCFPPVKENISQDIDHILETLSALAVDLGGTNLRVAIVSMKGEIVKKYTQFNPKTYEERINLILQMCVEAAAEAVKLNCRILGVGISTGGRVNPREGIVLHSTKLIQEWNSVDLRTPLSDTLHLPVWVDNDGNCAALAERKFGQGKGLENFVTLITGTGIGGGIIHQHELIHGSSFCAAELGHLVVSLDGPDCSCGSHGCIEAYASGMALQREAKKLHDEDLLLVEGMSVPKDEAVGALHLIQAAKLGNAKAQSILRTAGTALGLGVVNILHTMNPSLVILSGVLASHYIHIVKDVIRQQALSSVQDVDVVVSDLVDPALLGAASMVLDYTTRRIY

SEQ ID NO: 8 - protein, 722 aa, Homo sapiens
MEKNGNNRKLRVCVATCNRADYSKLAPIMFGIKTEPEFFELDVVVLGSHLIDDYGNTYRMIEQDDFDINTRLHTIVRGEDEAAMVESVGLALVKLPDVLNRLKPDIMIVHGDRFDALALATSAALMNIRILHIEGGEVSGTIDDSIRHAITKLAHYHVCCTRSAEQHLISMCEDHDRILLAGCPSYDKLLSAKNKDYMSIIRMWLGDDVKSKDYIVALQHPVTTDIKHSIKMFELTLDALISFNKQTLVLFPNIDAGSKEMVRVMRKKGIEHHPNFRAVKHVPFDQFIQLVAHAGCMIGNSSCGVREVGAFGTPVINLGTRQIGRETGENVLHVRDADTQDKILQALHLQFGKQYPCSKIYGDGNAVPRILKFLKSIDLQEPLQKKFCFPPVKENISQDIDHILETLSALAVDLGGTNLRVAIVSMKGEIVKKYTQFNPKTYEERINLILQMCVEAAAEAVKLNCRILGVGISTGGRVNPREGIVLHSTKLIQEWNSVDLRTPLSDTLHLPVWVDNDGNCAALAERKFGQGKGLENFVTLITGTGIGGGIIHQHELIHGSSFCAAELGHLVVSLDGPDCSCGSHGCIEAYASGMALQREAKKLHDEDLLLVEGMSVPKDEAVGALHLIQAAKLGNAKAQSILRTAGTALGLGVVNILHTMNPSLVILSGVLASHYIHIVKDVIRQQALSSVQDVDVVVSDLVDPALLGAASMVLDYTTRRIY

SEQ ID NO: 9 - protein, 722 aa, Homo sapiens
MEKNGNNRKLRVCVATCNRADYSKLAPIMFGIKTEPEFFELDVVVLGSHLIDDYGNTYRMIEQDDFDINTRLHTIVRGEDEAAMVESVGLALVKLPDVLNRLKPDIMIVHGDRFDALALATSAALMNIRILHIEGGEVSGTIDDSIRHAITKLAHYHVCCTRSAEQHLISMCEDHDRILLAGCPSYDKLLSAKNKDYMSIIRMWLGDDVKSKDYIVALQHPVTTDIKHSIKMFELTLDALISFNKRTLVLFPNIDAGSKEMVRVMRKKGIEHHPNFRAVKHVPFDQFIQLVAHAGCMTGNSSCGVREVGAFGTPVINLGTRQIGRETGENVLHVRDADTQDKILQALHLQFGKQYPCSKIYGDGNAVPRILKFLKSIDLQEPLQKKFCFPPVKENISQDIDHILETLSALAVDLGGTNLRVAIVSMKGEIVKKYTQFNPKTYEERINLILQMCVEAAAEAVKLNCRILGVGISTGGRVNPREGIVLHSTKLIQEWNSVDLRTPLSDTLHLPVWVDNDGNCAALAERKFGQGKGLENFVTLITGTGIGGGIIHQHELIHGSSFCAAELGHLVVSLDGPDCSCGSHGCIEAYASGMALQREAKKLHDEDLLLVEGMSVPKDEAVGALHLIQAAKLGNAKAQSILRTAGTALGLGVVNILHTMNPSLVILSGVLASHYIHIVKDVIRQQALSSVQDVDVVVSDLVDPALLGAASMVLDYTTRRIY

SEQ ID NO: 10 - protein, 722 aa, Homo sapiens
MEKNGNNRKLRVCVATCNRADYSKLAPIMFGIKTEPEFFELDVVVLGSHLIDDYGNTYRMIEQDDFDINTRLHTIVRGEDEAAMVESVGLALVKLPDVLNRLKPDIMIVHGDRFDALALATSAALMNIRILHIEGGEVSGTIDDSIRHAITKLAHYHVCCTRSAEQHLISMCEDHDRILLAGCPSYDKLLSAKNKDYMSIIRMWLGDDVKSKDYIVALQHPVTTDIKHSIKMFELTLDALISFNKRTLVLFPNIDAGSKEMVRVMRKKGIEHHPNFRAVKHVPFDQFIQLVAHAGCMIGNSSCGVREVGAFGTPVINLGTRQIGRETGENVLHVWDADTQDKILQALHLQFGKQYPCSKIYGDGNAVPRILKFLKSIDLQEPLQKKFCFPPVKENISQDIDHILETLSALAVDLGGTNLRVAIVSMKGEIVKKYTQFNPKTYEERINLILQMCVEAAAEAVKLNCRILGVGISTGGRVNPREGIVLHSTKLIQEWNSVDLRTPLSDTLHLPVWVDNDGNCAALAERKFGQGKGLENFVTLITGTGIGGGIIHQHELIHGSSFCAAELGHLVVSLDGPDCSCGSHGCIEAYASGMALQREAKKLHDEDLLLVEGMSVPKDEAVGALHLIQAAKLGNAKAQSILRTAGTALGLGVVNILHTMNPSLVILSGVLASHYIHIVKDVIRQQALSSVQDVDVVVSDLVDPALLGAASMVLDYTTRRIY

SEQ ID NO: 11 - protein, 722 aa, Homo sapiens
  VARIANT 436: note = X can be any naturally occurring amino acid
MEKNGNNRKLRVCVATCNRADYSKLAPIMFGIKTEPEFFELDVVVLGSHLIDDYGNTYRMIEQDDFDINTRLHTIVRGEDEAAMVESVGLALVKLPDVLNRLKPDIMIVHGDRFDALALATSAALMNIRILHIEGGEVSGTIDDSIRHAITKLAHYHVCCTRSAEQHLISMCEDHDRILLAGCPSYDKLLSAKNKDYMSIIRMWLGDDVKSKDYIVALQHPVTTDIKHSIKMFELTLDALISFNKRTLVLFPNIDAGSKEMVRVMRKKGIEHHPNFRAVKHVPFDQFIQLVAHAGCMIGNSSCGVREVGAFGTPVINLGTRQIGRETGENVLHVRDADTQDKILQALHLQFGKQYPCSKIYGDGNAVPRILKFLKSIDLQEPLQKKFCFPPVKENISQDIDHILETLSALAVDLGGTNLRVAIVSMKGEIVKKYTXFNPKTYEERINLILQMCVEAAAEAVKLNCRILGVGISTGGRVNPREGIVLHSTKLIQEWNSVDLRTPLSDTLHLPVWVDNDGNCAALAERKFGQGKGLENFVTLITGTGIGGGIIHQHELIHGSSFCAAELGHLVVSLDGPDCSCGSHGCIEAYASGMALQREAKKLHDEDLLLVEGMSVPKDEAVGALHLIQAAKLGNAKAQSILRTAGTALGLGVVNILHTMNPSLVILSGVLASHYIHIVKDVIRQQALSSVQDVDVVVSDLVDPALLGAASMVLDYTTRRIY

SEQ ID NO: 12 - protein, 722 aa, Homo sapiens
MEKNGNNRKLRVCVATCNRADYSKLAPIMFGIKTEPEFFELDVVVLGSHLIDDYGNTYRMIEQDDFDINTRLHTIVRGEDEAAMVESVGLALVKLPDVLNRLKPDIMIVHGDRFDALALATSAALMNIRILHIEGGEVSGTIDDSIRHAITKLAHYHVCCTRSAEQHLISMCEDHDRILLAGCPSYDKLLSAKNKDYMSIIRMWLGDDVKSKDYIVALQHPVTTDIKHSIKMFELTLDALISFNKRTLVLFPNIDAGSKEMVRVMRKKGIEHHPNFRAVKHVPFDQFIQLVAHAGCMIGNSSCGVREVGAFGTPVINLGTRQIGRETGENVLHVRDADTQDKILQALHLQFGKQYPCSKIYGDGNAVPRILKFLKSIDLQEPLQKKFCFPPVKENISQDIDHILETLSALAVDLGGTNLRVAIVSMKGEIVKKYTQFNPKTYEERINLILQMCVEAAAEAVKLNCRILGVGISTGGRVNPREGIVLHSTKLIQEWNSVDLRTPLSDTLHLPVWVDNDGNCAALAERKFGQGKGLENFVTLITGTGIGGGIIHQHESIHGSSFCAAELGHLVVSLDGPDCSCGSHGCIEAYASGMALQREAKKLHDEDLLLVEGMSVPKDEAVGALHLIQAAKLGNAKAQSILRTAGTALGLGVVNILHTMNPSLVILSGVLASHYIHIVKDVIRQQALSSVQDVDVVVSDLVDPALLGAASMVLDYTTRRIY

SEQ ID NO: 13 - protein, 722 aa, Homo sapiens
MEKNGNNRKLRVCVATCNRADYSKLAPIMFGIKTEPEFFELDVVVLGSHLIDDYGNTYRMIEQDDFDINTRLHTIVRGEDEAAMVESVGLALVKLPDVLNRLKPDIMIVHGDRFDALALATSAALMNIRILHIEGGEVSGTIDDSIRHAITKLAHYHVCCTRSAEQHLISMCEDHDRILLAGCPSYDKLLSAKNKDYMSIIRMWLGDDVKSKDYIVALQHPVTTDIKHSIKMFELTLDALISFNKRTLVLFPNIDAGSKEMVRVMRKKGIEHHPNFRAVKHVPFDQFIQLVAHAGCMIGNSSCGVREVGAFGTPVINLGTRQIGRETGENVLHVRDADTQDKILQALHLQFGKQYPCSKIYGDGNAVPRILKFLKSIDLQEPLQKKFCFPPVKENISQDIDHILETLSALAVDLGGTNLRVAIVSMKGEIVKKYTQFNPKTYEERINLILQMCVEAAAEAVKLNCRILGVGISTGGRVNPREGIVLHSTKLIQEWNSVDLRTPLSDTLHLPVWVDNDGNCAALAERKFGQGKGLENFVTLITGTGIGGGIIHQHELIHGSSFCAAELGHLVLSLDGPDCSCGSHGCIEAYASGMALQREAKKLHDEDLLLVEGMSVPKDEAVGALHLIQAAKLGNAKAQSILRTAGTALGLGVVNILHTMNPSLVILSGVLASHYIHIVKDVIRQQALSSVQDVDVVVSDLVDPALLGAASMVLDYTTRRIY

SEQ ID NO: 14 - protein, 722 aa, Homo sapiens
MEKNGNNRKLRVCVATCNRADYSKLAPIMFGIKTEPEFFELDVVVLGSHLIDDYGNTYRMIEQDDFDINTRLHTIVRGEDEAAMVESVGLALVKLPDVLNRLKPDIMIVHGDRFDALALATSAALMNIRILHIEGGEVSGTIDDSIRHAITKLAHYHVCCTRSAEQHLISMCEDHDRILLAGCPSYDKLLSAKNKDYMSIIRMWLGDDVKSKDYIVALQHPVTTDIKHSIKMFELTLDALISFNKRTLVLFPNIDAGSKEMVRVMRKKGIEHHPNFRAVKHVPFDQFIQLVAHAGCMIGNSSCGVREVGAFGTPVINLGTRQIGRETGENVLHVRDADTQDKILQALHLQFGKQYPCSKIYGDGNAVPRILKFLKSIDLQEPLQKKFCFPPVKENISQDIDHILETLSALAVDLGGTNLRVAIVSMKGEIVKKYTQFNPKTYEERINLILQMCVEAAAEAVKLNCRILGVGISTGGRVNPREGIVLHSTKLIQEWNSVDLRTPLSDTLHLPVWVDNDGNCAALAERKFGQGKGLENFVTLITGTGIGGGIIHQHELIHGSSFCAAELGHLVVSLDGPDCSCGSHGCTEAYASGMALQREAKKLHDEDLLLVEGMSVPKDEAVGALHLIQAAKLGNAKAQSILRTAGTALGLGVVNILHTMNPSLVILSGVLASHYIHIVKDVIRQQALSSVQDVDVVVSDLVDPALLGAASMVLDYTTRRIY

SEQ ID NO: 15 - protein, 722 aa, Homo sapiens
  VARIANT 615: note = X can be any naturally occurring amino acid
MEKNGNNRKLRVCVATCNRADYSKLAPIMFGIKTEPEFFELDVVVLGSHLIDDYGNTYRMIEQDDFDINTRLHTIVRGEDEAAMVESVGLALVKLPDVLNRLKPDIMIVHGDRFDALALATSAALMNIRILHIEGGEVSGTIDDSIRHAITKLAHYHVCCTRSAEQHLISMCEDHDRILLAGCPSYDKLLSAKNKDYMSIIRMWLGDDVKSKDYIVALQHPVTTDIKHSIKMFELTLDALISFNKRTLVLFPNIDAGSKEMVRVMRKKGIEHHPNFRAVKHVPFDQFIQLVAHAGCMIGNSSCGVREVGAFGTPVINLGTRQIGRETGENVLHVRDADTQDKILQALHLQFGKQYPCSKIYGDGNAVPRILKFLKSIDLQEPLQKKFCFPPVKENISQDIDHILETLSALAVDLGGTNLRVAIVSMKGEIVKKYTQFNPKTYEERINLILQMCVEAAAEAVKLNCRILGVGISTGGRVNPREGIVLHSTKLIQEWNSVDLRTPLSDTLHLPVWVDNDGNCAALAERKFGQGKGLENFVTLITGTGIGGGIIHQHELIHGSSFCAAELGHLVVSLDGPDCSCGSHGCIEAYASGMALQREAKKLHDEDLLLVEGMXVPKDEAVGALHLIQAAKLGNAKAQSILRTAGTALGLGVVNILHTMNPSLVILSGVLASHYIHIVKDVIRQQALSSVQDVDVVVSDLVDPALLGAASMVLDYTTRRIY

SEQ ID NO: 16 - protein, 722 aa, Homo sapiens
MEKNGNNRKLRVCVATCNRADYSKLAPIMFGIKTEPEFFELDVVVLGSHLIDDYGNTYRMIEQDDFDINTRLHTIVRGEDEAAMVESVGLALVKLPDVLNRLKPDIMIVHGDRFDALALATSAALMNIRILHIEGGEVSGTIDDSIRHAITKLAHYHVCCTRSAEQHLISMCEDHDRILLAGCPSYDKLLSAKNKDYMSIIRMWLGDDVKSKDYIVALQHPVTTDIKHSIKMFELTLDALISFNKRTLVLFPNIDAGSKEMVRVMRKKGIEHHPNFRAVKHVPFDQFIQLVAHAGCMIGNSSCGVREVGAFGTPVINLGTRQIGRETGENVLHVRDADTQDKILQALHLQFGKQYPCSKIYGDGNAVPRILKFLKSIDLQEPLQKKFCFPPVKENISQDIDHILETLSALAVDLGGTNLRVAIVSMKGEIVKKYTQFNPKTYEERINLILQMCVEAAAEAVKLNCRILGVGISTGGRVNPREGIVLHSTKLIQEWNSVDLRTPLSDTLHLPVWVDNDGNCAALAERKFGQGKGLENFVTLITGTGIGGGIIHQHELIHGSSFCAAELGHLVVSLDGPDCSCGSHGCIEAYASGMALQREAKKLHDEDLLLVEGMSVPKDEAVGALHLIQAVKLGNAKAQSILRTAGTALGLGVVNILHTMNPSLVILSGVLASHYIHIVKDVIRQQALSSVQDVDVVVSDLVDPALLGAASMVLDYTTRRIY

SEQ ID NO: 17 - protein, 722 aa, Homo sapiens; identical to SEQ ID NO: 2

SEQ ID NO: 18 - protein, 722 aa, Homo sapiens
MEKNGNNRKLRVCVATCNRADYSKLAPIMFGIKTEPEFFELDVVVLGSHLIDDYGNTYRMIEQDDFDINTRLHTIVRGEDEAAMVESVGLALVKLPDVLNRLKPDIMIVHGDRFDALALATSAALMNIRILHIEGGEVSGTIDDSIRHAITKLAHYHVCCTRSAEQHLISMCEDHDRILLAGCPSYDKLLSAKNKDYMSIIRMWLGDDVKSKDYIVALQHPVTTDIKHSIKMFELTLDALISFNKRTLVLFPNIDAGSKEMVRVMRKKGIEHHPNFRAVKHVPFDQFIQLVAHAGCMIGNSSCGVREVGAFGTPVINLGTRQIGRETGENVLHVRDADTQDKILQALHLQFGKQYPCSKIYGDGNAVPRILKFLKSIDLQEPLQKKFCFPPVKENISQDIDHILETLSALAVDLGGTNLRVAIVSMKGEIVKKYTQFNPKTYEERINLILQMCVEAAAEAVKLNCRILGVGISTGGRVNPREGIVLHSTKLIQEWNSVDLRTPLSDTLHLPVWVDNDGNCAALAERKFGQGKGLENFVTLITGTGIGGGIIHQHELIHGSSFCAAELGHLVVSLDGPDCSCGSHGCIEAYASGMALQREAKKLHDEDLLLVEGMSVPKDEAVGALHLIQAAKLGNAKAQSILRTAGTALGLGVVNILHTMNPSLVILSGVLASHYIHIVKDVIRQQALSSVQDVDMVVSDLVDPALLGAASMVLDYTTRRIY

SEQ ID NO: 19 - protein, 722 aa, Homo sapiens
MEKNGNNRKLRVCVATCNRADYSKLAPIMFGIKTEPEFFELDVVVLGSHLIDDYGNTYRMIEQDDFDINTRLHTIVRGEDEAAMVESVGLALVKLPDVLNRLKPDIMIVHGDRFDALALATSAALMNIRILHIEGGEVSGTIDDSIRHAITKLAHYHVCCTRSAEQHLISMCEDHDRILLAGCPSYDKLLSAKNKDYMSIIRMWLGDDVKSKDYIVALQHPVTTDIKHSIKMFELTLDALISFNKRTLVLFPNIDAGSKEMVRVMRKKGIEHHPNFRAVKHVPFDQFIQLVAHAGCMIGNSSCGVREVGAFGTPVINLGTRQIGRETGENVLHVRDADTQDKILQALHLQFGKQYPCSKIYGDGNAVPRILKFLKSIDLQEPLQKKFCFPPVKENISQDIDHILETLSALAVDLGGTNLRVAIVSMKGEIVKKYTQFNPKTYEERINLILQMCVEAAAEAVKLNCRILGVGISTGGRVNPREGIVLHSTKLIQEWNSVDLRTPLSDTLHLPVWVDNDGNCAALAERKFGQGKGLENFVTLITGTGIGGGIIHQHELIHGSSFCAAELGHLVVSLDGPDCSCGSHGCIEAYASGMALQREAKKLHDEDLLLVEGMSVPKDEAVGALHLIQAAKLGNAKAQSILRTAGTALGLGVVNILHTMNPSLVILSGVLASHYIHIVKDVIRQQALSSVQDVDVVVSDLVDPALLGAASTVLDYTTRRIY

SEQ ID NO: 20 - DNA, 21 bp, Homo sapiens
  source 1..21: mol_type = other DNA
agcacttcctggagtttgatg

SEQ ID NO: 21 - DNA, 24 bp, Homo sapiens
  source 1..24: mol_type = other DNA
atttgccttcgcagaaacacttga

SEQ ID NO: 22 - DNA, 20 bp, Homo sapiens
  source 1..20: mol_type = other DNA
gcccagagcatcttacgaac

SEQ ID NO: 23 - DNA, 18 bp, Homo sapiens
  source 1..18: mol_type = other DNA
gggtcccctggagcttgg

SEQ ID NO: 24 - protein, 20 aa, Homo sapiens
EAYASGMALQREAKKLHDED